(19) United States
(12) Patent Application Publication
(10) Pub. No.: US 2021/0267790 A1
(43) Pub. Date: Sep. 2, 2021

(54) Oral Dental Appliance for Improving Breathing

(71) Applicant: ARORA, LLC, Indianapolis, IN (US)

(72) Inventors: Michael Brock, Connersville, IN (US); J Groom, Oxford, OH (US); Brian Evans, Carmel, IN (US); Steven Green, Fishers, IN (US)

(21) Appl. No.: 17/323,105

(22) Filed: May 18, 2021

Related U.S. Application Data
(60) Provisional application No. 62/027,502, filed on Jul. 22, 2014.
(60) Provisional application No. 63/077,743, filed on Sep. 14, 2020.

(51) Int. Cl. A61F 5/56

(52) U.S. Cl. CPC A61F 5/566

(57) ABSTRACT

An oral dental appliance is provided that adjusts the position of a user's lower jaw relative to the upper jaw to avoid one cause of sleep apnea. The appliance includes an upper tray and a lower tray, each tray substantially identically configured and each tray defining a generally U-shaped channel to receive a bite guard molded to match the respective upper and lower dental arch of a user. The appliance further includes an adjustable clip assembly that is removably engageable to the upper and lower trays. The clip assembly is configured to be fixed in an adjustable forward (vestibular)-rearward (lingual) position relative to each other prior to engagement with the upper tray and the lower tray. The clip assembly is engageable to the trays so that the trays cannot move relative to the clips or relative to each other in the forward-rearward direction.

### PRIORITY CLAIM

This application is a utility filing from and claims priority to co-pending U.S. Provisional application No. 63/077,743, filed on Sep. 14, 2020, and U.S. Provisional application No. 63/027,502, filed on May 20, 2020. The entire disclosures of both provisional applications are incorporated herein by reference.

### BACKGROUND

The present disclosure relates to oral dental appliances and more particularly to a therapeutic mouthpiece to be worn during sleep.

Many people experience breathing problems, which may result in difficulty sleeping, in snoring, or in other more serious conditions such as obstructive sleep apnea. Sleep apnea is a sleep-related breathing disorder that is thought to affect between 1-10% of the adult population. Recent epidemiologic data indicate that 2% of women and 4% of men between the ages of 30 and 60 years meet the minimum diagnostic criteria for sleep apnea syndrome, representing more than 10 million individuals in the United States. Sleep apnea is a disorder with significant morbidity and mortality, contributing to increased risk of hypertension, cardiac arrhythmias, stroke, and cardiovascular death. Snoring is another common sleep-related breathing disorder which may be associated with or independent of sleep apnea.

One of the main reasons of the sleep disturbance is the relaxation of the tongue and pharyngeal walls to varying degrees during the several stages of sleep. When fully awake, these tissues have normal tone as air passes in and out of the lungs during respiration. However, during sleep, the musculature supporting these tissues relaxes. As air is inspired, the tongue and posterior walls of the pharynx collapse, causing snoring or, more seriously, causing partial or complete obstruction of the airway. Obstructive sleep apnea occurs due to a collapse of soft tissue within the upper airway during sleep. The ongoing force of inspiration serves to generate increasingly negative pressure within the pharynx, causing further collapse. The lack of respiration results in inadequate blood oxygenation, and rising carbon dioxide levels. The cardiovascular response produces an increase in the blood pressure and pulse. Cardiac arrhythmias often occur. The increase in carbon dioxide increase and oxygen desaturation triggers a transition to a lighter sleep stage, usually without wakefulness. This transition brings a return to tonicity of the muscles of the upper airway, allowing normal breathing to resume. The person then returns to deeper stages of sleep and the process is repeated. The disease is quantified in terms of respiratory disturbances per hour. Mild disease begins at ten per hour, but it is not uncommon to find patients with indices ranging from sixty events to over one hundred or more per hour.

Not surprisingly, sleep is extremely fragmented and of poor quality in persons suffering from sleep apnea. As a result, such persons typically feel tired upon wakening and may fall asleep at inappropriate times during the day. All aspects of quality of life, from physical and emotional health, to social functioning are impaired by obstructive sleep apnea.

One treatment for such breathing disorders involves the use of devices inserted into a user's mouth for extending the user's lower jaw forward, possibly in conjunction with a face mask for supplying air to the user. These devices open the breathing passageway more fully to allow easier breathing through the nose and mouth. Such appliances or devices for improving breathing have included upper and lower arches that are connected together outside a user's mouth and then inserted into the user's mouth as an integrated unit to position the user's lower jaw forward. Although these devices may treat some breathing problems, many of these devices do not sufficiently treat more serious conditions such as obstructive sleep apnea, because the devices do not allow the user or a clinical professional to easily and accurately adjust the position of the user's lower jaw to achieve optimum positioning. Moreover, many of these devices are often unwieldy and uncomfortable for the user with respect to insertion into the user's mouth and subsequent use after insertion into the user's mouth, resulting in minimal usage. As a result of these and other deficiencies, there remains a need for an oral dental appliance that is simple to use and comfortable to wear.

### SUMMARY OF THE DISCLOSURE

An oral dental appliance is provided that adjusts the position of a user's lower jaw relative to the upper jaw to avoid one cause of sleep apnea. The appliance includes an upper tray and a lower tray, each tray substantially identically configured and each tray defining a generally U-shaped channel to receive a bite guard molded to match the respective upper and lower dental arch of a user. The bite guard can be molded into each tray or can be introduced to the tray. The appliance further includes an adjustable clip assembly that is removably engageable to the upper and lower trays. The clip assembly is configured to be fixed in an adjustable forward (vestibular)-rearward (lingual) position relative to each other prior to engagement with the upper tray and the lower tray. The clip assembly is engageable to the trays so that the trays cannot move relative to the clips or relative to each other in the forward-rearward direction.

In one feature, the trays are identical so that any tray can be the upper tray or the lower tray. In a further feature, each of the trays includes a pair of wings that are pivotable towards each other in a direction transverse to the forward-rearward direction. This allows the trays to be “one size fits all”, with adjustments for mouth size made by pivoting the wings of the trays to an appropriate position.

In another aspect of the appliance, each tray includes a front wall arranged to be positioned adjacent the lips of the user and an opposite rear wall arranged to be positioned adjacent the tongue of the user when the dental appliance is worn in the mouth of the user. The adjustable clip assembly includes an upper clip removably engagable with the upper tray, and including a rear end arranged to be positioned adjacent the rear wall of the upper tray when the upper clip is engaged with the upper tray, and a lower clip removably engageable with the lower tray and including a rear end arranged to be positioned adjacent the rear wall of the lower tray when the upper clip is engaged with the lower tray. A fastener is provided for connecting the upper clip and the lower clip and for fastening the upper clip and the lower clip together. When the upper clip and the lower clip are connected by the fastener, the clips are configured for movement relative to each other in the forward-rearward direction to the adjustable forward-rearward position. The fastener is then operable to fasten the clips together at the forward-rearward position. The clips are then engaged to the respective trays so that the respective trays cannot move relative to the clips or relative to each other in the forward-rearward direction.

The clips are configured for a simple snap-fit engagement with the respective trays. In one feature, the clips and trays can be configured to permit relative movement between a tray and its associated clip in a direction transverse to the forward-rearward direction.

An adjustment mechanism is provided that simplifies the adjustment of the forward-rearward position of the two clips. The mechanism is provided on the lid of a case configured to store the clip assembly. One of the clips is held by the lid of the case and the other clip is held by a separate tool, although the two clips are connected by the fastener. The tool is mechanically coupled to an indicator needle that is pivotably mounted on the lid so that small movements of the tool to provide fine forward-rearward adjustments to the clips translates to larger, more perceptible, movements of the indicator needle. The lid includes a dial with indicia indicative of the forward-rearward position of the clips relative to each other.

### DESCRIPTION OF THE DRAWINGS

FIG. 1 is a rear perspective view of an oral dental appliance according to the present disclosure.

FIG. 2 is front perspective view of the appliance shown in FIG. 1.

FIG. 3 is an exploded view of the appliance shown in FIG. 1.

FIG. 4 is top view of the upper and lower clips view of the appliance shown in FIG. 1, with the clips engaged at different travel positions.

FIG. 5 is side cross-sectional view of view of the appliance shown in FIG. 1, shown with the upper and lower clips assembled at the different travel positions of FIG. 4.

FIG. 6 is a bottom perspective view of the tray of the appliance shown in FIG. 1.

FIG. 7 is a rear perspective view of the an oral dental appliance according to a further embodiment of the present disclosure.

FIG. 8 is front perspective view of the appliance shown in FIG. 7

FIG. 9 is further rear perspective view of the appliance shown in FIG. 7.

FIG. 10 is top view of the upper tray of the appliance shown in FIG. 7.

FIG. 11 is a top view of the upper tray shown in FIG. 10 with the wings of the tray in different positions.

FIGS. 12a-12b are upper and side exploded views of the upper and lower clips of the appliance shown in FIG. 7.

FIGS. 13a-13b are side cross-sectional views of the assembled upper and lower clips shown in FIGS. 12a-12b, respectively, with the clips in different relative forward and rearward positions.

FIGS. 14a-14b are rear views of the assembled upper and lower clips shown in FIGS. 12-13, with the clips in different relative lateral positions.

FIG. 15 is an exploded view of upper and lower clips according to a further embodiment for use with the appliance shown in FIG. 7.

FIG. 16 is an assembled view of the upper and lower clips shown in FIG. 15.

FIG. 17 is a side cross-sectional view of the assembled upper and lower clips shown in FIG. 16.

FIGS. 18a-18b are side cross-sectional views of the appliance of FIG. 7 with the assembled upper and lower clips shown in FIG. 17, with the clips in different forward and rearward relative positions.

FIG. 19 is an exploded view of upper and lower clips according to another embodiment for use with the appliance shown in FIG. 7.

FIG. 20 is a perspective view of an embedded nut of the lower clip shown in FIG. 19.

FIG. 21 is a side cross-sectional view of the assembled upper and lower clips shown in FIG. 19.

FIG. 22 is a top perspective view of the assembled upper and lower clips shown in FIG. 21.

FIG. 23 is an exploded view of a container and the dental appliance of the previous figures.

FIG. 24 is a perspective view of the complete container shown in FIG. 23 with a clip adjustment mechanism.

FIG. 25 is an exploded view of the container and clip adjustment mechanism shown in FIG. 24.

FIG. 26 is a top view of the container and clip adjustment mechanism shown in FIG. 24.

FIG. 27 is a perspective view of the container and clip adjustment mechanism of FIG. 24 shown with the multi-tool of the mechanism removed.

FIG. 28 is a partially exploded view showing the multi-tool being added to the container shown in FIG. 27.

FIG. 29. is a perspective view of the assembled container and clip adjustment mechanism of the present disclosure.

FIG. 30 is a perspective view of a tool for use with the clip adjustment mechanism shown in FIG. 29.

FIG. 31 is a top view of the multi-tool of the clip adjustment mechanism shown in the previous figures.

FIG. 32 is a top view of the clip adjustment mechanism with the assembled clips in a first position.

FIG. 33 is a top view of the clip adjustment mechanism with the assembled clips in a second position.

FIG. 34 is a top view of the clip adjustment mechanism with the assembled clips in a third position.

FIG. 35 is a top view of the clip adjustment mechanism with the assembled clips in a fourth position.

FIG. 36 is a top view of the clip adjustment mechanism with the assembled clips in a fifth position.

FIG. 37 is a perspective view of a tool of the multi-tool used with assembled clips to disengage the clips from the trays.

FIG. 38 is perspective view of a container and clip adjustment mechanism according to a further embodiment.

FIG. 39 is a top view of the clip adjustment mechanism shown in FIG. 38.

FIG. 40 is an exploded view of the container and clip adjustment mechanism shown in FIG. 39.

FIG. 41 is sequence of views showing the assembly and use of container and adjustment mechanism shown in FIG. 38.

FIG. 42 is a side view of a clip assembly and a top view of the adjustment mechanism showing one adjustment of the clip assembly in which the lower bite is moved forward by 2 mm.

FIG. 43 is a side view of a clip assembly and a top view of the adjustment mechanism showing another adjustment of the clip assembly in which the lower bite is moved forward 3 mm.

FIG. 44 is a side view of a clip assembly and a top view of the adjustment mechanism showing a further adjustment of the clip assembly in which the lower bite is moved forward 5 mm.

FIG. 45 is a side view of a clip assembly and a top view of the adjustment mechanism showing an adjustment of the clip assembly in which the lower bite is moved forward a maximum amount of 7 mm.

FIG. 46 is a side view of a clip assembly and a top view of the adjustment mechanism showing an adjustment of the clip assembly in which the upper and lower bite are even or neutral.

FIG. 47 is a side view of a clip assembly and a top view of the adjustment mechanism showing an adjustment of the clip assembly in which the lower bite is moved rearward a maximum amount of 3 mm.

FIG. 48 is a perspective view of the multi-tool of the clip adjustment mechanism shown in FIG. 31 in use on the upper tray of an appliance.

FIG. 49 is a perspective view of the multi-tool in engagement with the upper tray to disconnect the upper tray from the upper clip.

### DETAILED DESCRIPTION

For the purposes of promoting an understanding of the principles of the disclosure, reference will now be made to the embodiments illustrated in the drawings and described in the following written specification. It is understood that no limitation to the scope of the disclosure is thereby intended. It is further understood that the present disclosure includes any alterations and modifications to the illustrated embodiments and includes further applications of the principles disclosed herein as would normally occur to one skilled in the art to which this disclosure pertains.

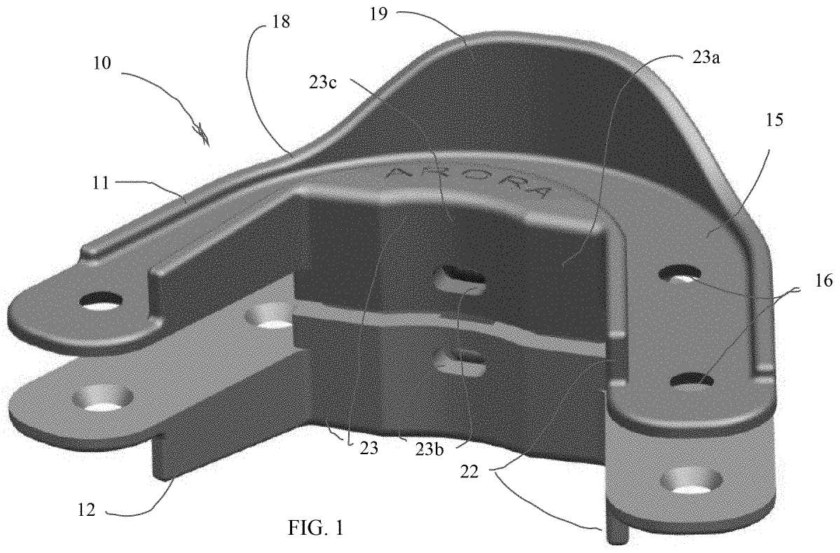

An oral dental appliance 10 according to the present disclosure includes identically configured generally U-shaped upper and lower trays 11, 12, as shown in FIGS. 1-3. The trays define a generally U-shaped channel 15 that is configured to receive corresponding bite guards that are heat-moldable to conform to either the upper or lower row of teeth or dental arch. The moldable material can be a thermoplastic resin or an elastomer polymer, such as EVA (ethylene vinyl acetate). It is understood that the bite guards can be formed directly by the patient biting into the moldable material, or can be formed based on a separate mold of the patient's upper and lower dental arches.

The tray defines openings 16 in the channel 15 to receive the moldable material used to form the bite guard so that the subsequently added bite guard is mechanically fixed to the appliance 10. In one approach, the bite guard is molded into the trays as the dental arch configurations are formed. In another approach, the bite guard is pre-formed and then heated to be pressed into the trays. In accordance with one aspect of the dental appliance 10, the upper and lower trays are identical, since the unique attributes of the user's upper and lower dental arches are accommodated by the moldable bite guard. This feature assures a comfortable fit of the appliance in the user's mouth, which is essential to ensure user compliance every night.

The tray includes a front wall 18 and an opposite rear wall 22 that define the channel 15. The front wall 18 includes an enlarged panel 19 that covers the gums of the user when the appliance is properly positioned. The enlarged panel prevents irritation to the lips that might result from moving across an exposed edge of the appliance. It is contemplated that the trays 11, 12 are formed of a typical dental plastic, such as a hard acrylic or biocompatible polymer, that can accept the moldable bite guard as the bite guard is heated and formed, without itself deforming. The material is sufficiently rigid to holds its shape after repeated use, but soft enough not to irritate the soft tissues of the user's mouth. It is contemplated that the trays can be formed by a conventional molding process. In order to avoid irritation to the patient's mouth, the edges and corners of the trays can be rounded.

The rear wall 22 defines a dome portion 23 that allows proper shaping and control of the heat-moldable material of the bite guard while preventing intrusion of the material outside the channel 15. The back wall 23a of the dome portion extends below the channel 15 and defines an opening 23b that is used to lock a clip component in place, as described further herein. The back wall 23a can include a recess 23c to provide clearance for the user's tongue when the appliance is worn.

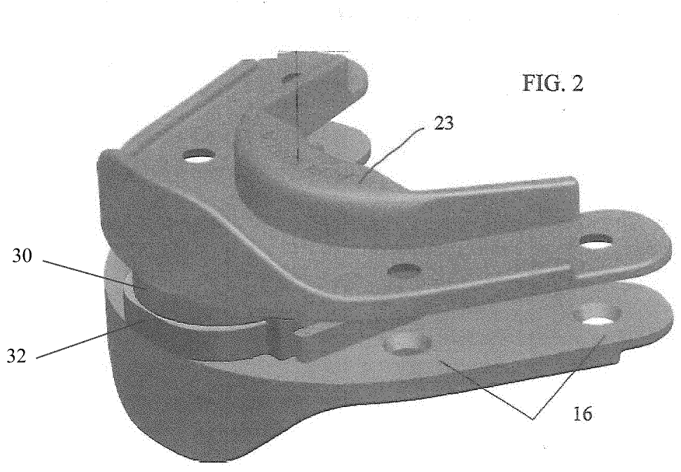

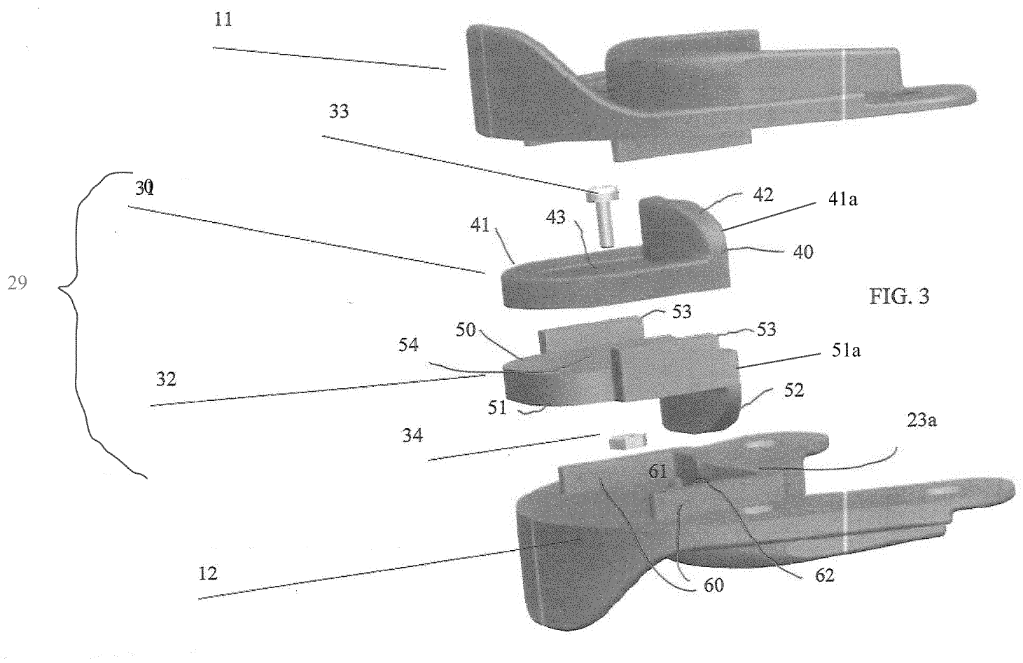

The appliance 10 includes an adjustable clip assembly 29 that is removably engageable to the two trays. The assembly includes an upper clip 30 and a lower clip 32 that are removably engaged within the respective upper and lower tray 11, 12, as shown in FIGS. 2-3. The clips are configured to permit relative adjustment of the upper and lower trays in the vestibular and lingual directions, which in turn can adjust the position of the lower jaw forward (vestibular) or backward (lingual) relative to the upper jaw. As shown in more detail in FIGS. 3-6, the two clips are generally L-shaped in side view. The upper clip 30 comprises a body 40 that forms a plate portion 41 and a tongue 42 perpendicular to the plate at a rear or lingual end 41a of the plate portion. The plate portion 41 defines a slot 43 that is elongated in the forward-rearward (vestibular-lingual) direction of the plate portion when the clip is mounted within the upper tray 11.

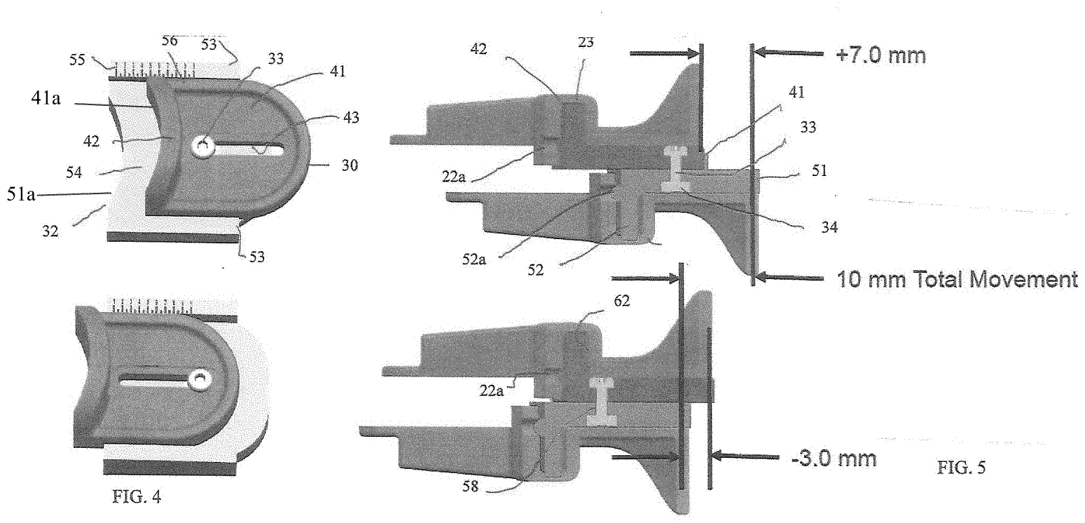

The lower clip 32 comprises a body 50 that forms a plate portion 51 and a perpendicular tongue 52 at a rear or lingual end 51a of the plate portion. The plate portion 51 includes side walls 53 that define a channel 54 which receives the plate portion 41 of the upper clip 30 in nested engagement, as best seen in FIG. 4. The upper clip 30 can slide within the channel 54 of the lower clip 32 to adjust the relative forward-rearward positions of the two clips. One or both of the side walls 53 of the lower clip 32 can include an adjustment scale 55 (FIG. 4) that provides a visual indication of the relative position of the two clips, and therefore the two trays that carry the clips. An indicator arrow 56 on the plate portion 41 of the upper clip 30 can be aligned with a particular tic mark on the scale 55 to assist the user in adjusting the appliance 10, as discussed in more detail herein.

The two clips 30, 32 are fastened together by a bolt 33 that extends through the slot 43 of the upper clip 30 and through a central bore 58 in the lower clip 32, although it can be appreciated that the slot and bore can be reversed between the two clips. A nut 34 is threaded onto the bolt to fasten the two clips together. The central bore 58 of the lower clip can include a recess sized to receive the nut 34 and hold it against rotation. In one embodiment the nut can be over-molded into the lower clip 32 when the clip is formed so that the nut is embedded in the clip. In another embodiment, the central bore 58 can be threaded so that a separate nut is not required. Either approach allows the user to easily tighten the two clips together by simply rotating the bolt 33 without the need to hold the nut in position. The bolt can have a head configured to receive a conventional driving tool, such as a hex head to receive a hex wrench.

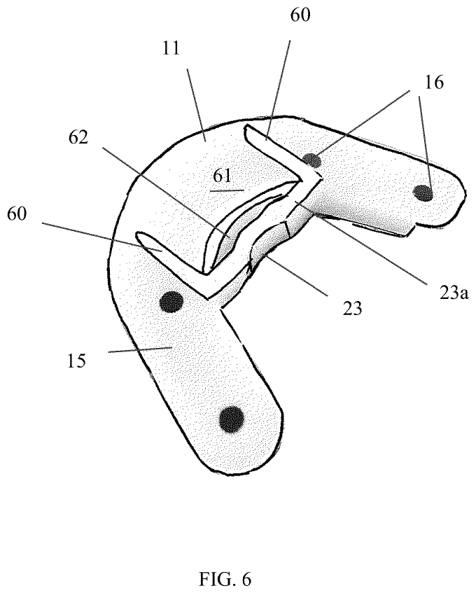

Returning to FIG. 3, the trays 11, 12 include a opposite walls 60 that integrate with the back wall 23a of the dome portion to form a niche 61 sized to receive one of the clips 30, 32. The tray further defines a cavity 62 inside the dome portion 23 of the tray that is configured to receive the tongue 42, 52 of one of the clips 30, 32, as depicted in FIGS. 5-6. A knob 42a, 52a on the respective tongues 42, 52 is engaged within an opening 22a defined in the back wall 23a of the dome 23 to releasably lock the tongue in place. More specifically, the interface between the knob 42a, 52a and the opening 22a holds the upper tray 11 and lower tray 12 in position on the assembled clips 30, 32. It can be appreciated that the knob 42a, 52a and opening 22a form a snap-fit connection between the respective clips and trays.

The manner of assembly and adjustment of the appliance 10 can be appreciated from considering FIGS. 4-5. In an initial step, the user mounts the upper clip 32 on the lower clip 32 by seating the plate portion 41 of the upper clip in the channel 54 of the lower clip. The bolt 33 can be passed through the slot 43 and into the bore 58 in the lower clip to engage the nut 34. The bolt is initially loosely fastened to the nut (or to the threaded bore) so that the upper clip 30 can slide relative to the lower clip 32. The user positions the upper clip so that the indicator arrow 56 points to a proper number on the adjustment scale 55. The physician or clinician can indicate to the user the proper scale number to place the upper clip relative to the lower clip. It is contemplated that when a person starts using the appliance 10 the appropriate position of the lower jaw relative to the upper jaw is not known. In other words, it is not known how far the lower jaw needs to be moved forward in order to address the user's breathing problem, or more specifically how far forward the lower jaw needs to move to prevent the soft tissue collapse that causes obstructive sleep apnea. Consequently, the clinician proposes an initial adjustment number on the scale 55 that can be readily understood by the user. After a few nights at the initially prescribed adjustment number, the clinician can recommend moving the upper clip forward if the soft tissue collapse is still present, or moving the upper clip rearward if the lower jaw position is uncomfortable or painful for the use. Again, the clinician provides the user with a number on the scale and the user can easily align the clips so that the indicator arrow 56 points to the proper number. This process can be interactive and continue over several days until an ideal adjustment number is reached. The user then knows to orient the upper clip at that ideal adjustment number each time the appliance is used.

Once the proper orientation of the upper clip and lower clip has been set, the user tightens the bolt 33 to lock the two clips together in that orientation. The upper tray 11 can now be engaged to the upper clip 30 by pushing the tongue 42 into the cavity 62 of the upper tray until the knob 42a locks in the opening 22a. The knob essentially snaps into the opening so that no special tools are required to mate the tray with the clip. Likewise, the lower tray 12 can be locked onto the lower clip 32 in a similar manner so that the assembled appliance appears as in the cross-section of FIG. 5 and in the perspective views of FIGS. 1 and 2. It can be appreciated that the complete appliance will include a bite guard, formed into the respective upper and lower dental arch, with the bite guard integrated into the respective upper and lower trays 11, 12, although the guards are not depicted in the figures. The user then places the appliance in his/her mouth with the upper and lower dental arches seated within the associated bite guards. When the dental arches are properly seated, the appliance will automatically adjust the forward/rearward position of the user's lower jaw.

As shown in FIG. 5, the present appliance 10 provides a ten mm (10 mm) range of forward/rearward (vestibular/lingual) adjustable positions of the lower tray relative to the upper tray. In one specific embodiment, the appliance is configured so that the lower tray can be moved seven mm (7 mm) forward from a neutral position in which the user's upper and lower jaws are in their relaxed position. The lower tray can then be moved three mm (3 mm) rearward from that neutral position. These forward and rearward positions can be correlated to a particular number on the adjustment scale 55. For instance, the maximum forward position (7 mm) can be assigned a scale number “9”, while the maximum rearward position (−3 mm) can be assigned the scale number “1”. Other scale numbering or identification schemes are contemplated that simplify the adjustment process for the user.

The appliance 10 can be completely and easily disassembled for regular cleaning. The upper and lower trays 11, 12 “click” into connection with the two clips 30, 32, so the trays can be readily “un-clicked” from the clips. This “click” connection also ensures that the trays can be readily separated from the assembled appliance if it becomes necessary for the user to fully open his/her mouth. The separated tray can be easily reconnected to the appliance, even while the tray is engaged on the user's teeth. The upper and lower clips 30, 32 are connected by a bolt and nut. The bolt can be easily loosened and removed using a simple tool, such as an Allen wrench provided to the user with the appliance.

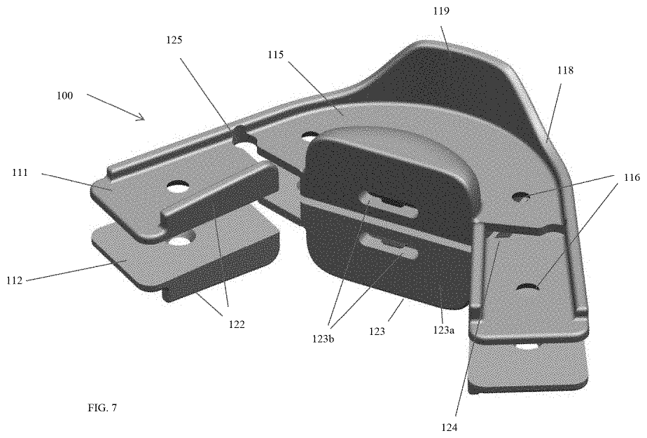

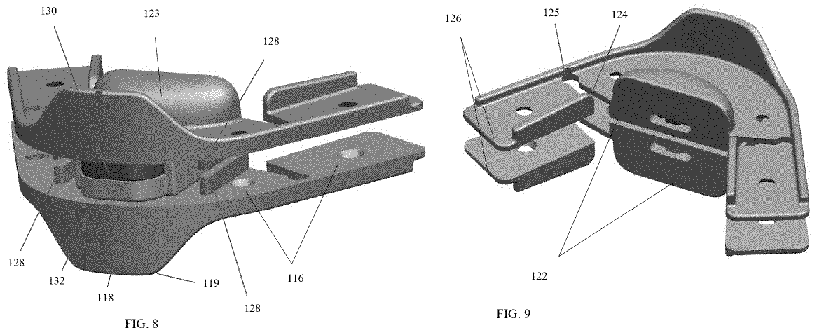

In another embodiment, the dental appliance 100, shown in FIGS. 7-11, includes upper and lower trays 111, 112, respectively, that are modified from the trays in the previous embodiment. The trays 111, 112 each define a channel 115 with openings 116 for receiving the moldable material, as described above. Each tray includes a front wall 118 with an enlarged portion 119, a rear panel 122 and dome 123 similar to the like components in the previous embodiment. The dome 123 allows proper shaping of the moldable material while retaining the material on the tray 111, 112, even as the material is molded around the user's teeth. The dome 123 includes a back wall 123a that defines an oval opening 123b that is elongated in the transverse direction for reasons discussed below. The underside of the trays includes a pair of guide walls 128 (FIG. 8) that receive the clips 130, 132. It is understood that the trays 111, 112 can be identically configured and simply flipped over with the guided walls facing each other, as shown in the drawings, to form the appliance 100.

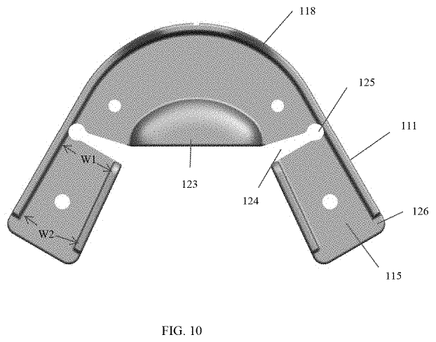

The trays 111, 112 include a slot 124 that extends from an inner edge of the tray to a notch 125 adjacent the front wall 118 of the tray. In one embodiment, the notch 125 impinges slightly into the front wall, as best seen in FIGS. 7, 10. The slot 124 thus defines an interior wing 126 at each side of the tray 111. In one aspect, each wing 126 has a width W1 at the front of the wing that is less than the width W2 at the rear bite section of the wing (FIG. 10). The greater width allows additional moldable material to be provided to receive the larger molars.

Each notch 125 operates as a hinge to allow each wing 126 to be bent inward in the plane of the channel 115 (or transverse to the forward-rearward direction) toward the forward body 111a, as illustrated in the sequence shown in FIGS. 11a-c. In the baseline configuration in FIG. 11a the slot 124 defines an angled gap between the wing 126 and the forward body 111a. The wing 126 can be pivoted or bent toward the forward body to narrow the slot 124′, as shown in FIG. 11b and bent even more until the wing contacts the body 111a and the slot is closed, as shown in FIG. 11c. The wings 126 can be pivoted to a range of positions to adjust to the particular dental arches of the user. As noted above, the two trays 111, 112 are identical in construction to reduce manufacturing costs and simplify replacement of a tray. In one specific embodiment, the trays 111, 112 can be molded from a polymer, such as nylon or PMMA (polymethyl methacrylate), that can be bent or flexed without failure as the wings are pivoted. The polymer material can permanently deform when the wings are pivoted about the notch 125 to eliminate any spring-back of the wings during sizing of the trays. The polymer material must also be capable of accepting the moldable material placed on the trays when the assembled appliance is fitted to the user's mouth and bite. It can be appreciated that this feature of the dental appliance 100 allows the trays 111,112 to accommodate different sizes of dental arches, with the baseline position of FIG. 11a corresponding to the largest dental arch and the position of FIG. 11c corresponding to the smallest dental arch.

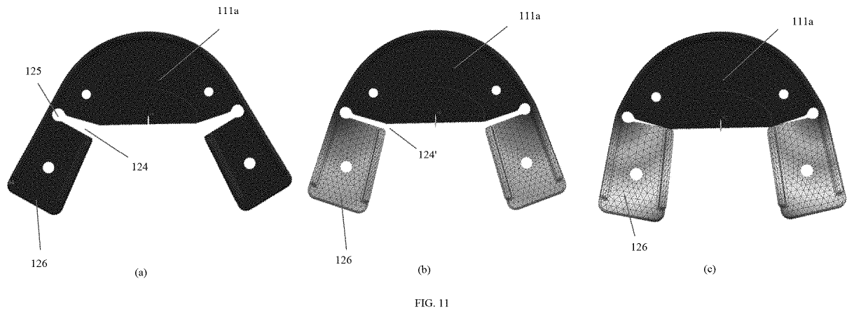

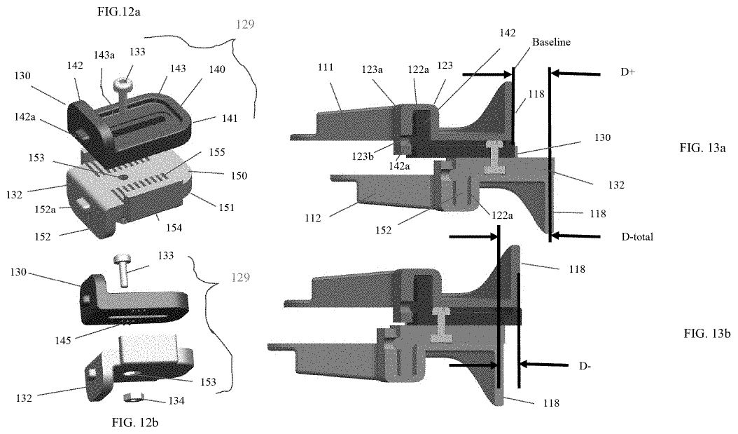

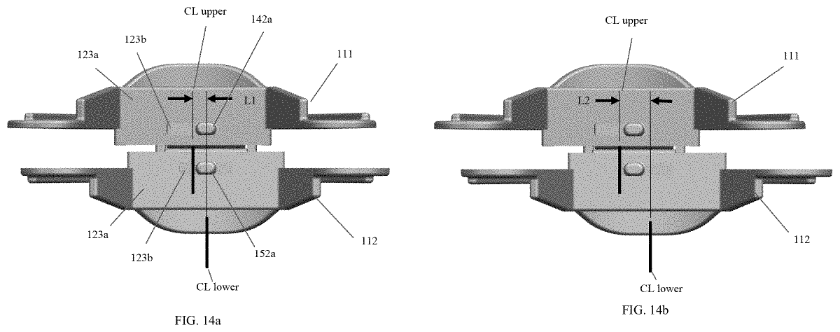

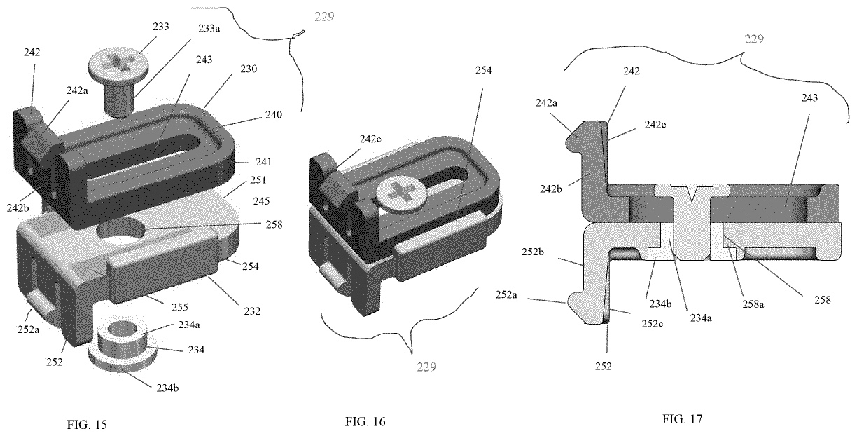

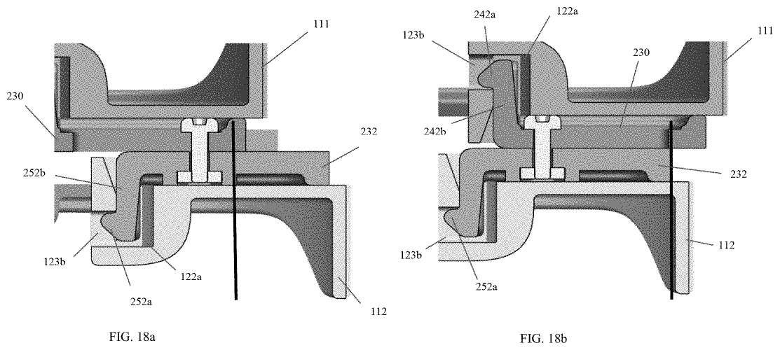

As shown in FIG. 13a-13b, the trays 111, 112 are removably engageable to a clip assembly 129 which includes corresponding upper and lower clips 130, 132, as illustrated in FIGS. 12a-12b. The upper clip 130 includes a body 140 molded to form a plate 141 and a tongue 142 projecting generally perpendicularly from the plate. The tongue 142 is configured to be received in an opening 122a defined in the dome 123, as shown in FIG. 13a. The plate 141 includes a slot 143 that is elongated in the front-back direction. The slot can include a recess 143a around the perimeter of the slot to receive the head of bolt 133. The tongue 142 includes a projection or knob 142a projecting perpendicularly from the tongue in the front-back direction. The knob 142a is seated within the opening 123b in the rear panel 122, as shown in FIGS. 13a-14b. The knob and opening form a snap-fit connection, as described above.

The lower clip 132 is differently constructed from the upper clip. The lower clip 132 includes a body 150 that is molded to form a plate 151 and a tongue 152, similar to the plate and tongue of the upper clip. The tongue 152 is configured to fit within an opening 122a in the lower tray 112, as shown in FIG. 12a. The tongue 152 includes a rearward projecting knob 152a, similar to the knob 142a, that is seated within the elongated opening 123b in the lower tray. The plate 151 defines a bore 153 that is aligned with the slot 143 in the upper tray when the two clips are mated. The lower clip 132 includes channel walls 154 that are arranged to receive the upper clip, preventing relative lateral movement between the two clips while permitting longitudinal or front-back relative movement of the clips. The upper surface of the plate 151 defines a series of lateral ridges 155 (FIG. 12a) that mate with corresponding ridges 145 on the underside of the plate 141 of the upper clip (FIG. 12b). The ridges 155, 145 interlock to help fix the relative front-back position of the two clips. The ridges 151 on the lower clip can also act as a relative position indicator, similar to the adjustment scale 55 of the appliance 10. The upper surface of plate 151 can thus be provided with indicia corresponding to each of the ridges, similar to the adjustment scale 55.

The two clips 130, 132 are engaged to each other by a bolt 133 and nut 134. The bolt extends through the slot 143 and bore 153 to engage the nut 134. It is contemplated that the slot and bore can be reversed between the two clips. When the two clips are in their desired orientation, the bolt and nut can be tightened to fix the clips together. As shown in FIGS. 13a-13b, the slot 143 permits a range of relative movement D-total between the two trays. The front face of the front wall 118 of the upper tray 111 can define a baseline or neutral position in which the front wall of the upper tray is aligned with the front wall of the lower tray. The lower tray can move forward relative to the upper tray a distance D+ and rearward a distance D− that is less than the forward distance D+. In a specific embodiment, the two trays can be configured for a total distance D-total of 10 mm, with a forward distance D+ of 7 mm and a rearward distance D− of 3 mm. As explained above, this relative movement between the two trays moves the movable lower jaw relative to the fixed upper dental arch. The two clips can be adjusted independent of the two trays to establish the desired relative movement D+, D− once the trays are mounted on the tongues 142, 152 of the clips.

As best seen in FIGS. 14a-14b, the knobs 142a, 152a of the clips 130, 132 are sized to fit within the elongated opening 123b of the respective tray 111, 112. The knob can be elongated with a width less than the width of the opening 123b to allow the knob 142a, 152a to travel along the length of the opening. The knob and elongated opening thus allow the two trays to move laterally relative to each other so that their respective centerlines, CL upper and CL lower, can be offset. The two trays can be offset a dimension L1 by moving one tray, in this case upper tray 111, to one side until the knob 142a is at one end of the opening, while the other knob, in this case knob 152a, remains at the centerline CL lower. The two trays can be moved to a maximum lateral offset dimension L2 by moving one knob (knob 142a) to the right end of the opening in one tray (111), and the other knob (knob 152a), to the left end of the opening in the other tray (tray 112). In one specific embodiment, each slot has a length of 2.0 mm so that the dimension L1 is 2.0 mm and the maximum dimension L2 is 4.0 mm. It is noted that this relative lateral offset generally has no effect on the snoring or apnea treatment, but is instead provided to accommodate the anatomy of the user's jaw and bite. It is contemplated that in some embodiments the opening 123b is sized to the knobs 142a, 152a so that no relative lateral movement is permitted between the trays 111, 112.

Another embodiment of a clip assembly 229 is shown in FIGS. 15-18b. The assembly 229 includes an upper clip 230 having a body 240 that is molded to form a plate 241 and a tongue 242. The plate includes a slot 243 and ridges 245 similar to these components in the clip 130 described above. Likewise, the lower clip 232 includes a body 250 molded to form a plate 251 and tongue 252 with a bore 258 and ridges 255, similar to the clip 132. In one difference from the previous embodiment, the threaded shank 233a of a bolt 233 is engaged to a modified nut 234. The nut includes a threaded collar 234a that is seated in the bore 258 and an enlarged flange 234b that is seated in a recess 258a of the bore. The nut 234 is thus embedded within the plate 251 of the lower clip 232. The nut 234 can be over-molded into the body 250 when the lower clip 232 is formed or can be affixed within the bore 258 and recess 258a in a known manner.

In another deviation from the previous clips, the upper and lower clips 230, 232 include a projection or latch 242a, 252a at the end of an arm 242b, 252b disposed in a U-shaped notch 242c, 252c formed in the tongue 242, 252. The latch 242a, 252a projects outward from the tongue 242, 252, as best seen in FIG. 17, and the arm 252b, 252b is resiliently deflectable so that the latch can be deflected forward toward the plate 241, 251. The latch 242a, 252a is elongated similar to the knob 142a, 152a so that latch can fit within the elongated opening 123b in the same manner as the knobs, as depicted in FIGS. 18a-18b, to positively hold the respective tray and clip together. In other words, the latches 242a, 252a and respective openings 123b form a snap-fit connection, as described above. The latches 242a, 252a can move along the length of the respective opening 123b to permit adjustment of the relative lateral position of the two trays 111, 112. The two clips 230, 232 also permit the front-back adjustment, as shown in FIG. 22. When the tongues 242, 252 of the clips are placed into the openings 122a of the two trays, the latches engage the openings 123b to effectively lock the clips and trays in engagement. The trays can be removed from the clips by pushing the latch 252a forward to bend the arm 242b, 252b.

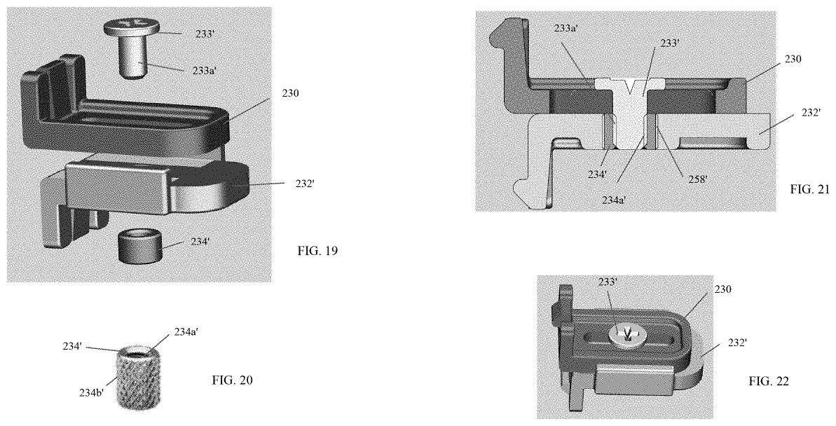

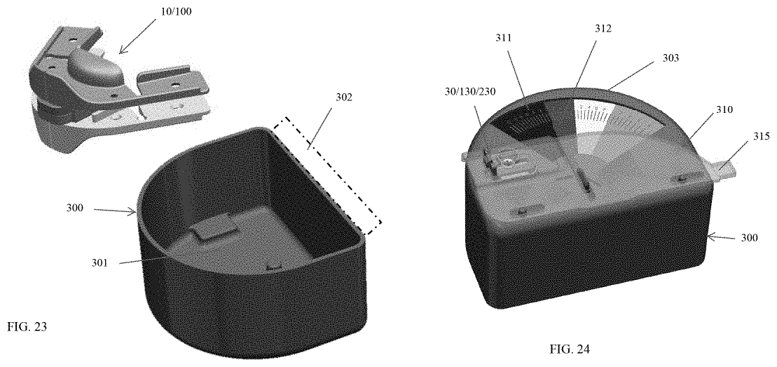

A variation of these clips is shown in FIGS. 19-22. The upper tray 230 is unchanged, but the lower tray 232′ is modified to accommodate an embedded nut 234′. In particular, the lower tray includes a bore 258′ that receives the nut 234′ in in press-fit engagement. The nut 234′ can have a knurled outer surface 234b′, as shown in FIG. 20. to enhance the engagement within the bore. The bolt 233′ includes a threaded stem 233a′ that engages the threaded bore 234a′ of the embedded nut. The two trays 230, 232′ are adjustably positioned and fastened with the bolt 233′ in the manner described above.

In another aspect of the present disclosure, an apparatus is provided for aligning the upper and lower trays 11/12, 111/112 by adjusting the relative locations of the upper and lower clips 31/32, 131/132, 231/232 before mounting the assembled clips within the respective trays. As discussed above, treatment provided by the dental appliances disclosed herein adjusts the position of the lower jaw relative to the stationary upper jaw, typically by moving the lower jaw forward. The amount of movement depends on the comfort level of the user and on the effectiveness of a particular position to treat the user's condition. In the embodiment of FIGS. 3-4, the relative position of the upper and lower clips 31, 32 is achieved by aligning the upper clip with markings 55 on the lower clip. This approach can be difficult and confusing to some users. The present disclosure contemplates a clip adjustment mechanism that is simple to use and that provides an accurate indication of the relative location of the two clips prior to being tightened together. The mechanism is incorporated into a container 300 for the dental appliance 10, 100, as shown in FIGS. 23-29. The base 301 of the container provides a space sized to snugly receive the loosely assembled appliance. A cover 303 is mounted to the base by a hinge 302, such as a living hinge. The top of the cover 303 is provided with an adjustment scale 311 with dimension numbers and marks positioned in a radial sweep on the cover. An indicator or needle 312 is movable to point to a particular mark to indicate the actual space between the upper and lower clips. The adjustment mechanism 310 further includes a multi-tool 315 that is configured to move the upper clip 30/130/230 relative to the lower clip and to simultaneously move the needle 312.

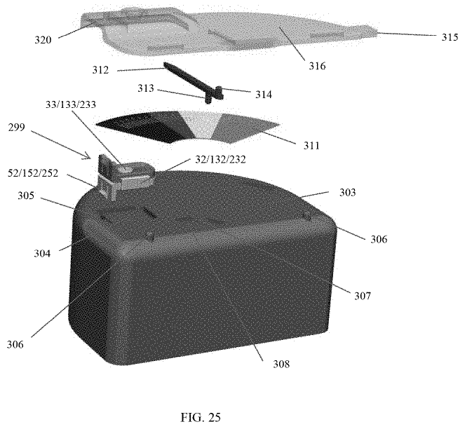

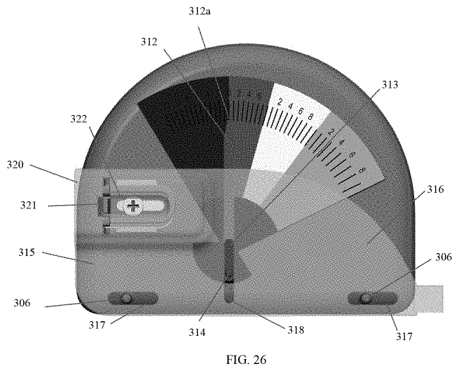

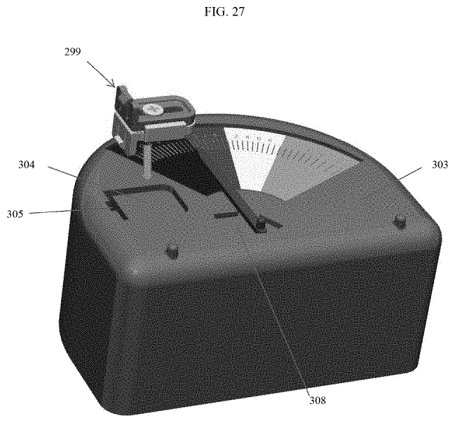

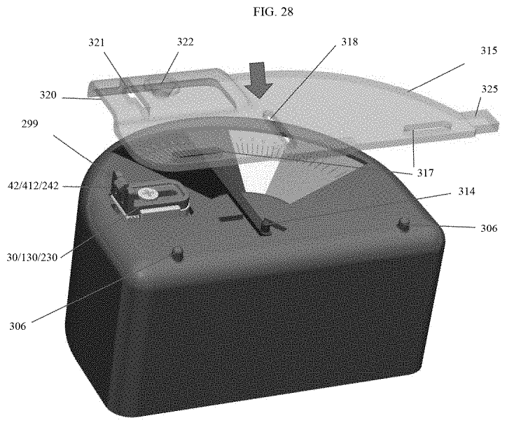

As shown in FIG. 25, the indicator needle 312 includes a downward directed pivot post 313 that is seated within a pivot bore 307 defined in the cover 303. The needle also includes an upward directed guide post 314 that that is seated within a slot 318 defined in the plate 316 of the multi-tool 315 (FIG. 26). The indicator needle is mounted to the cover 303 with the pivot post 313 seated within the pivot bore 307 and with the adjacent portion of the needle situated within a recess 308 defined in the cover. The walls of the recess contain the needle 312 and provide a limit to its angular rotation.

The surface of the cover 303 further defines a recess 304 for receiving the clip assembly 299 (FIG. 25, 27, 28). A slot 305 in the recess receives the tongue 52/152/252 of the lower clip 32/132/232 so that the lower clip is held against movement by the cover 303. On the other hand, the upper clip 30/130/230 is free to move once the bolt 33/133/233 is loosened.

The adjustment mechanism includes the multi-tool 315 that is in the form of a molded plate 316 that is sized to be seated on the top surface of the cover 303 of the container 300. The plate 316 defines a pair of guide slots 317 that are elongated on a common axis, or at least along parallel axes, as shown in FIG. 26. The cover 303 includes a pair of spaced-apart guide pins 306 that extend through the slots 317 when the plate 315 is mounted on the cover. The guide pins thus restrain the movement of the multi-tool 315 in one direction, namely back and forth across the cover. The plate 316 further defines an indicator guide slot 318 that receives the guide post 314 of the indicator needle 312. The guide slot 318 is oriented perpendicular to the direction of movement of the plate 316 or perpendicular to the axis between the two guide posts 306. As demonstrated in FIGS. 32-36, lateral movement of the plate 316 causes the indicator needle 312 to pivot on its pivot post 313 which is offset upward from the needle guide post 314. The distance between the needle guide post 314 and the pivot post 323 is much less than the distance between the tip 312a of the indicator needle and the pivot post 323. In one embodiment, the distance between the tip and the pivot post is at least three times the distance between the pivot post and the guide post. This ratio of distances provides a mechanical advantage that converts slight movements of the multi-tool 315 into readily discernible movements of the indicator needle. It can be appreciated that fine adjustments of the clip assembly 299 can be as small as one millimeter. A one-millimeter movement can be difficult to discern. However, a three-millimeter movement of the needle tip 312a can be seen relatively easily by most users.

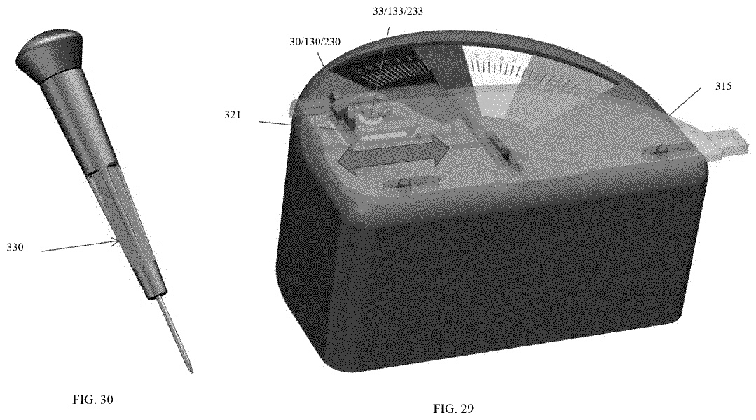

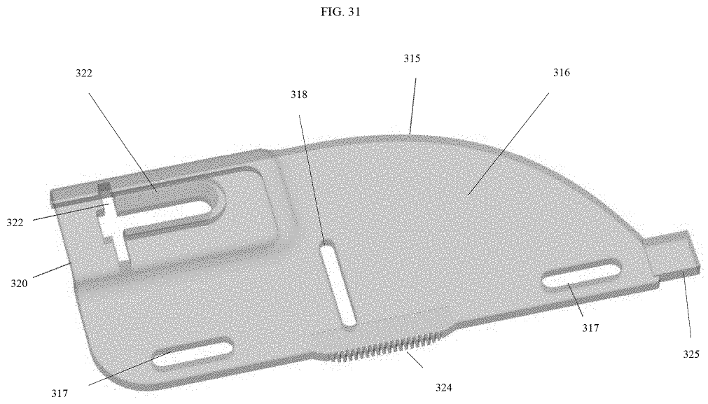

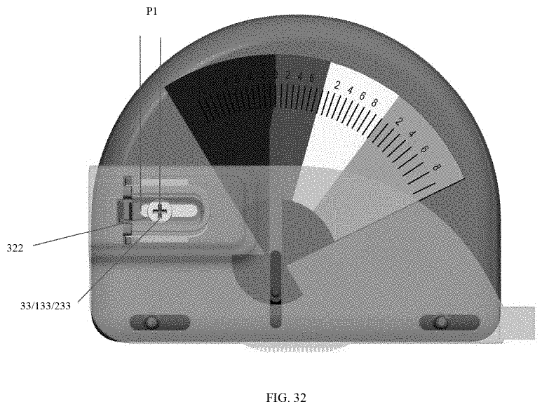

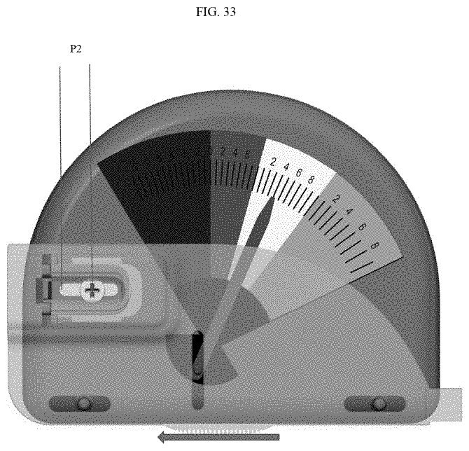

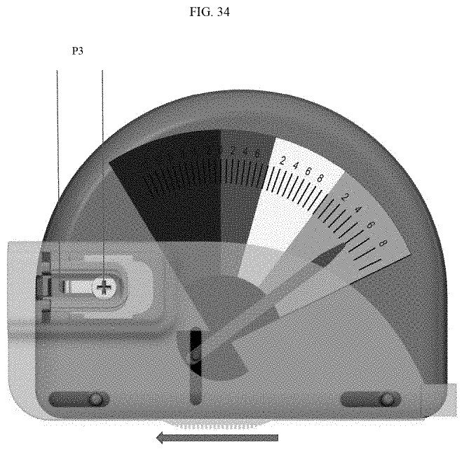

The plate 316 defines a housing 320 for receiving the upper clip when it is mounted in the recess 308 in the cover, as best seen in FIGS. 28, 29 and 31. The housing includes a slot 321 to receive the tongue 42/142/242 of the upper clip 30/130/230. The engagement between the tongue and the slot forces the upper clip to move laterally with the multi-tool 315, as illustrated in FIG. 29. The plate further defines a slot 322 through which the head of the bolt is accessible to loosen and tighten the bolt and thus the assembled clips.

To use the clip adjustment mechanism 310, the clip assembly 299 are placed within the recess 308 in the cover 303 of the container, with the tongue of the lower clip seated within the slot 305 and the body of the lower clip within the recess, as depicted in FIG. 27. The multi-tool 315 is then placed on the cover with the posts 306 projecting through the guide slots 317, the needle guide post 314 projecting through the slot 318 and the tongue of the upper clip extending through the slot 321, as depicted in FIGS. 28-29. With the loosely assembled clip contained between the multi-tool 315 and the cover, the multi-tool can be moved laterally as needed to adjust the position of the upper clip relative to the lower clip. The multi-tool 315 can be provided with a ridged edge 324 (FIG. 31) to facilitate manual movement of the plate 316. Once the desired position has been established, a tool 330 can be used to tighten the bolt 33/133/233 thereby fixing the upper and lower clips together. The multi-tool 315 can be removed and the properly adjusted and tightened clips can be removed and placed between the upper and lower trays.

The range of adjustments of the clip assembly 299 is illustrated in FIGS. 32-36. In FIG. 32, the position of the bolt relative to the end of the slot is identified as the baseline position P1 in which the upper and lower clips are aligned, as shown in FIG. 16 for instance. In position P1 the tip of the indicator needle points to the numeral “0” to indicate that the clip assembly is in its baseline configuration. In FIG. 33, the multi-tool is moved slightly to the left so that the bolt is farther from the left end of the slot in position P2. In this position, the indicator needle is pointing to the numeral “4” in a “yellow” zone of the scale 311. It is noted that in the scale in this embodiment, differently colored zones are provided to help the user properly position the assembled clips. In the example in FIG. 33, a specialist may tell the user to adjust the multi-tool until the needle is pointing to “yellow 4”. The specialist understands that “yellow 4” corresponds to a particular offset in which the lower tray, and thus the user's jaw, is moved forward, 3 mm for instance. It can be appreciated that the left movement of the upper clip relative to the fixed lower clip corresponds to moving the lower clip and lower tray forward when the dental appliance is worn in the user's mouth because the upper jaw is fixed while the lower jaw can move forward and rearward.

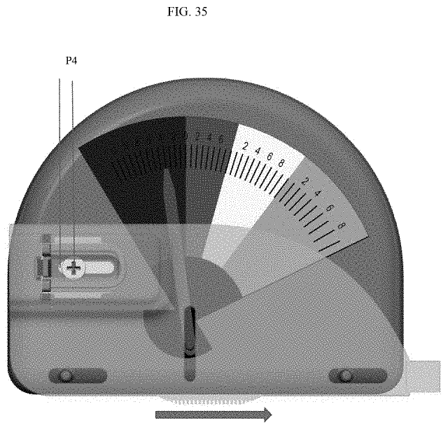

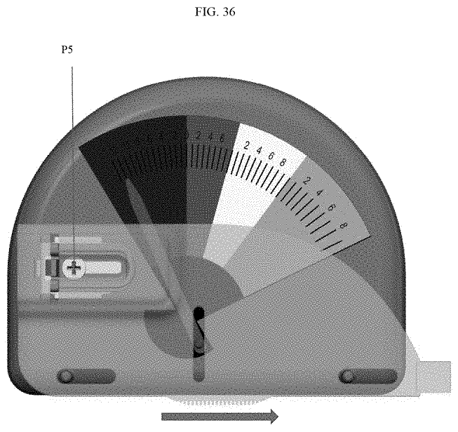

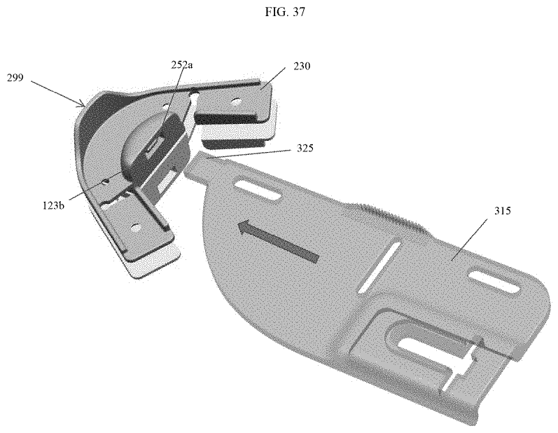

Further movement of the multi-tool to the left moves the upper clip to the position P3, which corresponds to the numeral “6” in the green segment of the scale, as shown in FIG. 34. In this position, the offset between the upper and lower clips is nearly at its maximum, such as a 6 mm forward movement of the user's jaw. Again, the specialist can instruct the user to move the multi-tool until the needle reaches “green 6” corresponding to a particular forward offset of the lower jaw that may be beneficial to the user, such as 6 mm. A movement of the multi-tool to the right is shown in FIG. 35 in which the upper clip is in position P4, corresponding to “blue 4”, which can correspond to a movement of 1 mm. In this position, the upper tray is offset inward relative to the lower tray, such as shown in FIG. 5. More particularly, this corresponds to moving the lower jaw inward relative to the upper jaw. In FIG. 36, the upper tray has been moved to its maximum inward position P5, such as 3 mm.

The multi-tool 315 includes a tool 325 that is used to dislodge the trays from the clips. In particular, the tool 325 can be pushed into the opening 123b of a respective tray to press against the latch 252a of the respective clip 230, 232 shown in FIGS. 15-22. It is contemplated that a new user of the dental appliance will need to adjust the tray positions as needed for comfort and effectiveness. In order to make the necessary adjustment the assembled clips need to be removed from the trays, or more accurately the trays need to be removed from the assembled clips. The tool 325 on the multi-tool simplifies the process of disengaging the tray from the clip.

The multi-tool 315 may be molded from a resilient plastic. Preferably the material of the multi-tool is transparent or translucent to make it easier for the user to mount the multi-tool on the cover and over a clip assembly 299. The container 300 can be formed of a harder plastic than the multi-tool and may be preferably opaque to protect the stowed dental appliance from sunlight.

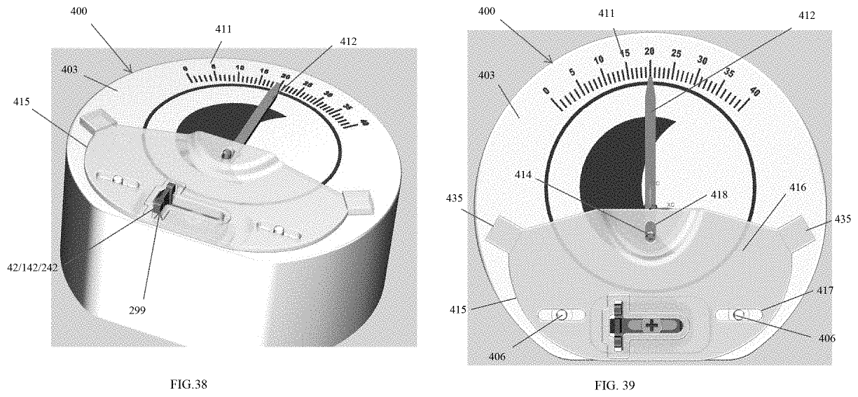

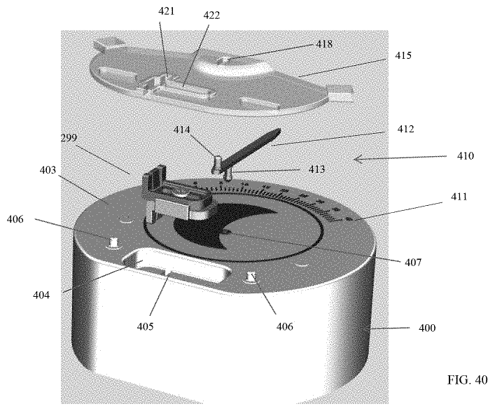

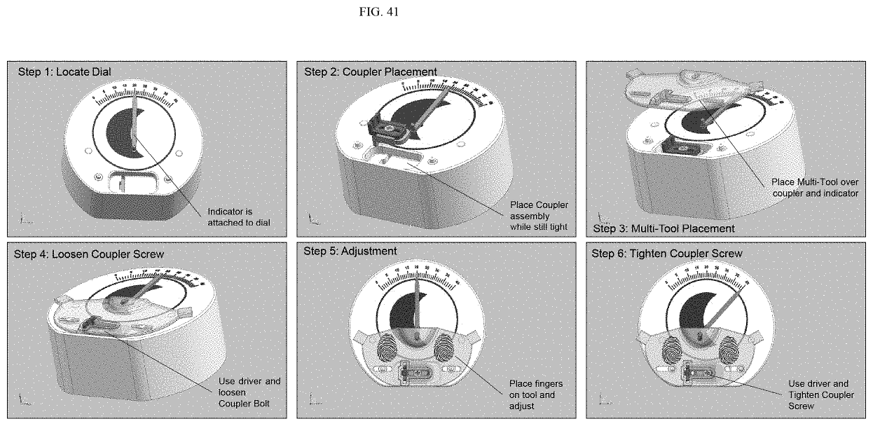

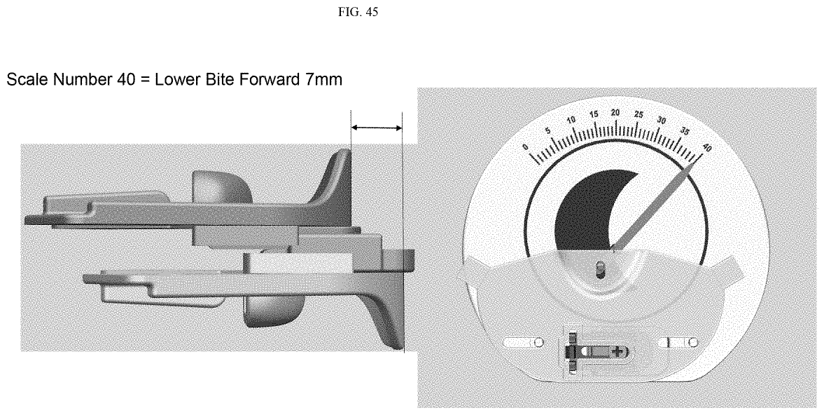

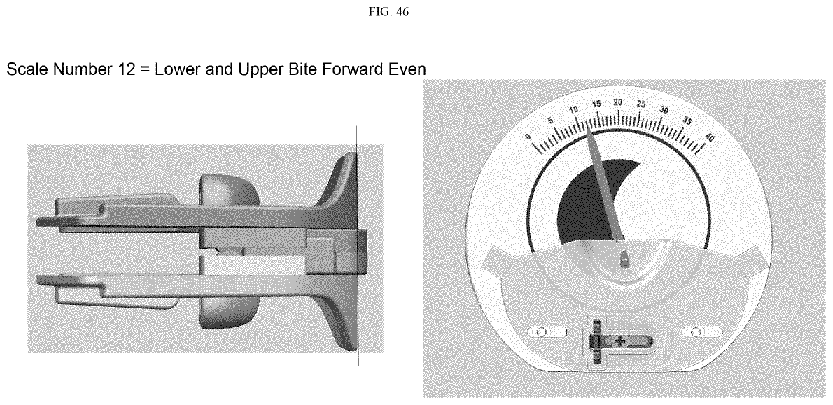

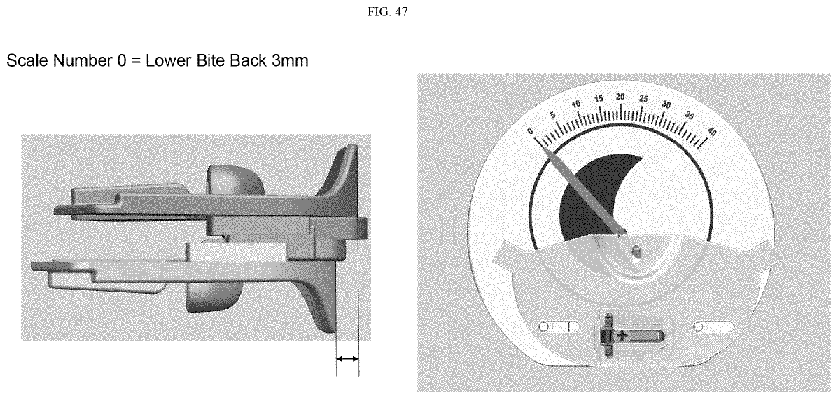

Another embodiment of the container and adjustment mechanism is shown in FIGS. 38-47. A case 400 can be configured like the case 300 to contain an accessory. The case 400 includes a cover 403 that supports a clip adjustment mechanism 410 that includes a dial 411 and a pivoting indicator needle 412. As best seen in FIG. 39, the dial 411 includes a graduated scale of numbers ranging from 0 to 40. As with the adjustment mechanism 310, the mechanism 410 is configured to magnify the slight relative movements of the upper tray of a clip assembly 299 into larger movements of the needle 412 on the dial 411 so that the user can easily visualize the movements.

As best seen in FIG. 40, the indicator needle 412 includes a pivot post 413 that is rotatably seated in the pivot bore 407 defined in the cover 403 of the case. The needle also includes a guide post 414 that is received within a guide slot 418 of a multi-tool 415. In contrast to the indicator needle 312 of the mechanism 310, the guide pin 414 is between the pivot axis of the indicator needle 412 and the clip assembly 299 being adjusted. However, the end result is the same—the relative locations of the pivot post and guide post allow multiplication of the lateral movement of the upper clip into a larger rotational movement of the indicator needle 412.

In contrast to the multi-tool 315 of the previous embodiment, the multi-tool 415 does not include a coupler housing. Instead, the cover 403 defines a recess 404 with a tongue slot 405 to receive the clip assembly 299. The plate 416 of the multi-tool is seated over the recess and the assembled clips within the recess. The plate defines a tongue slot 421 to receive the tongue 42/142/242 of the upper clip and an opening 422 for access to the attachment bolt, as in the previous embodiment. The plate also defines guide slots 417 that are seated over guide posts 406 projecting from the cover 413, as in the previous embodiment. An indicator guide slot 418 in the plate receives the guide post 414 of the indicator needle, again as in the previous embodiment of the case 300.

FIG. 41 illustrates the steps in assembling and using the case 400 and clip adjustment mechanism 410. In the first step, the indicator needle is mounted on the cover with the pivot post 413 disposed within the pivot bore 407. The assembled clips are then placed within the recess 404 with the tongue of the lower clip seated within the slot 405. The multi-tool 415 is placed over the assembled clips with the guide slots aligned with the respective guide posts. It is contemplated that some manipulation of the indicator needle 412 may be required to engage the indicator guide slot 418 in the plate while also engaging the tongue of the upper clip within the tongue slot 421 of the plate. Once the multi-tool is in position, the fixation bolt of the assembled clips is loosened using the tool 330 to allow the upper clip to translate relative to the fixed lower clip. The user can move the plate from side-to-side by placing two fingers on the plate, as illustrated in Step 5. In this step the user can move the multi-tool plate from until the indicator needle is pointing to the desired scale number on the dial 411. It can be appreciated that since the tongue of the upper clip is lodged within the plate, the upper clip automatically moves with the plate, but its movement is smaller than the movement of the indicator needle due to the lever arm created by between the pivot post and guide post of the indicator needle. Once the indicator needle is pointing to the desired setting, the user can use a tool, such as the tool 330 to tighten the fixation bolt and clamp the assembled clips together.

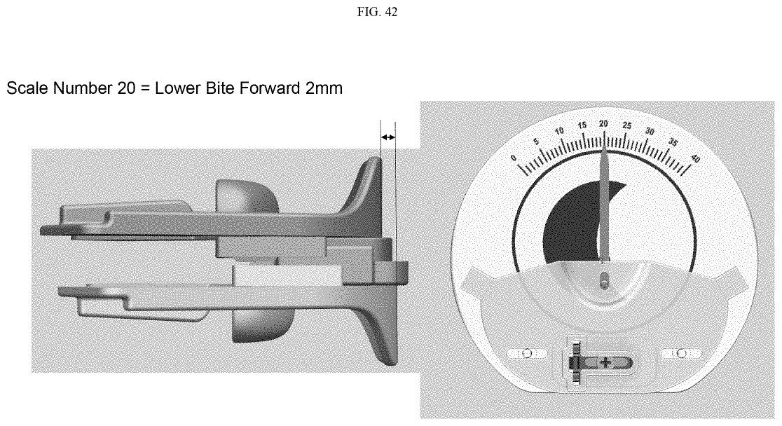

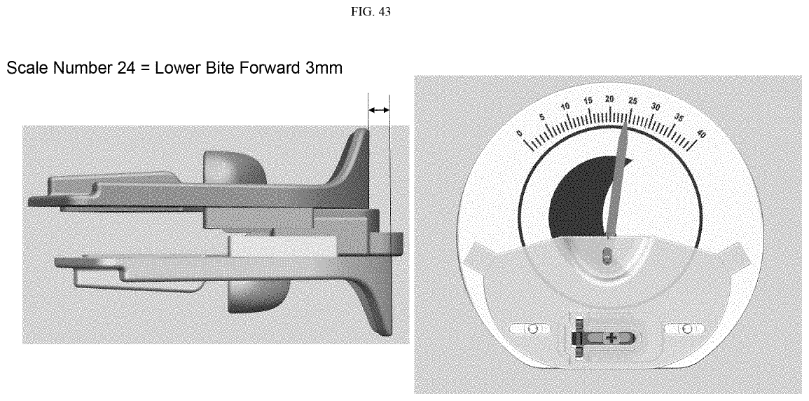

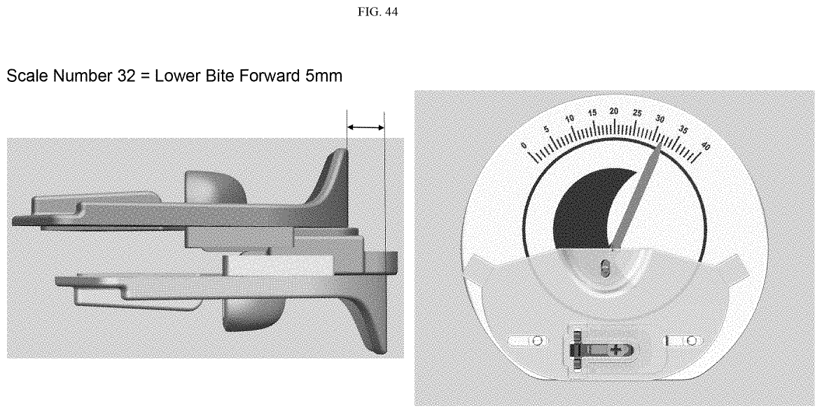

FIGS. 42-47 show the relative positions of the upper and lower clips corresponding to a particular scale number on the dial 411. Thus, in the present embodiment, a 2 mm forward bite movement, meaning that the lower tray projects 2 mm forward relative to the upper tray, corresponds to a scale number of 20. A 5 mm forward bite movement corresponds to a scale number of 24, while a 5 mm movement corresponds to a scale number of 32. The maximum forward movement permitted with the present embodiment, 7 mm, has a scale number of 40. When the two clips are aligned or in their neutral orientation, the scale number is 12. With this arrangement of scale numbers, a rearward bite movement still corresponds to a positive number on the dial. The maximum rearward bite movement, 3 mm in the present embodiment, corresponds to a scale number of 0. It can thus be seen that the 10 mm range of bite movement achieved by the appliances 10/100/200 of the present disclosure generates a scale number range from 0 to 40, meaning that a 1 mm relative movement of the clips corresponds to 4 scale marks on the dial 411, or conversely that a 0.25 mm movement of the upper clip corresponds to one scale mark on the dial 411. It has been found that this configuration of the clip adjustment mechanism 410 allows the use to make finer gradation adjustments, namely 0.25 mm. It is contemplated that other ranges of adjustments can be made by modifying the needle 412, and particularly the relative positions of the pivot post 413 and the guide post 414, as well as the relative positions of the clip recess 404 and pivot bore 407.

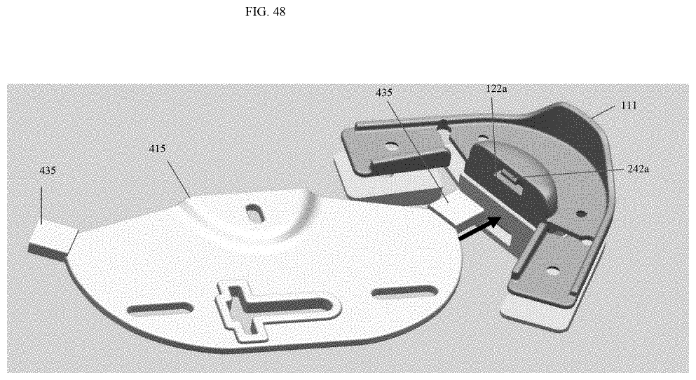

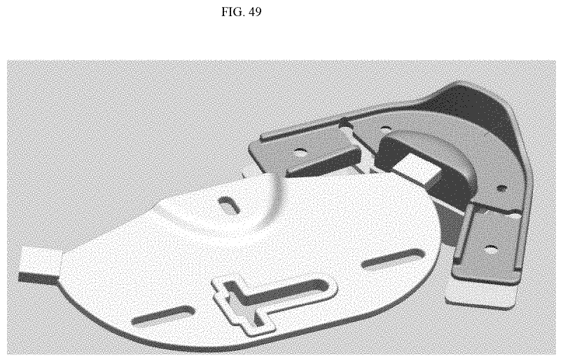

The multi-tool 415 includes two tools 435 that can be used to disengage a tray from a clip, as shown in FIGS. 48-49. As described above with respect to the multi-tool 315, the tools 435 of the multi-tool 415 can pushed into the opening 122a in a tray, such as tray 111 in FIGS. 48-49, to engage the latch 242a. Pushing the tool 435 further into the opening disengages the latch from the opening, allowing the tray to be separated from the clip.

The present disclosure should be considered as illustrative and not restrictive in character. It is understood that only certain embodiments have been presented and that all changes, modifications and further applications that come within the spirit of the disclosure are desired to be protected.

What is claimed is:

1. An oral dental appliance comprising:
an upper tray and a lower tray, each tray substantially identically configured and each tray defining a generally U-shaped channel to receive a bite guard molded to match the respective upper and lower dental arch of a user; and
an adjustable clip assembly removably engageable to said upper tray and said lower tray, said clip assembly configured to be fixed in an adjustable forward-rearward position relative to each other prior to engagement with said upper tray and said lower tray,
wherein the clip assembly is engageable to the trays so that said trays cannot move relative to the clips or relative to each other in a forward-rearward direction.

2. The oral dental appliance of claim 1, wherein each of said trays includes a pair of wings that are pivotable towards each other in a direction transverse to said forward-rearward direction.

3. The oral dental appliance of claim 1, wherein said channel of each of the trays includes a plurality of holes therethrough to receive material of the bite guard embedded therein.

4. The oral dental appliance of claim 1, wherein:
each tray includes a front wall arranged to be positioned adjacent the lips of the user and an opposite rear wall arranged to be positioned adjacent the tongue of the user when the dental appliance is worn in the mouth of the user; and
said adjustable clip assembly includes;
an upper clip removably engagable with said upper tray, and including a rear end arranged to be positioned adjacent said rear wall of said upper tray when the upper clip is engaged with said upper tray;
a lower clip removably engageable with said lower tray and including a rear end arranged to be positioned adjacent said rear wall of said lower tray when the upper clip is engaged with said lower tray; and
a fastener for connecting said upper clip and said lower clip and for fastening said upper clip and said lower clip together,
wherein when said upper clip and said lower clip are connected by said fastener, said upper clip and said lower clip are configured for movement relative to each other in said forward-rearward direction to said adjustable forward-rearward position,
wherein said fastener is operable to fasten the clips together at said forward-rearward position, and
wherein the clips are engageable to the respective trays so that the respective trays cannot move relative to the clips or relative to each other in said forward-rearward direction.

5. The dental appliance of claim 4, wherein;
said upper and lower clip each include a plate and a tongue projecting from said plate at said rear end thereof; and
the upper and lower trays each include a cavity configured to removably receive said tongue of a respective one of the clips,
whereby said upper and lower tray are fixed against movement in said forward-rearward direction relative to each other when the tongue of said upper and lower clips is engaged in the cavity of the respective one of said trays.

6. The oral dental appliance of claim 5, wherein:
one of said clips defines a pair of spaced apart side walls; and
said plate of the other of said clips is sized to be nested between said side walls to permit relative movement in said forward-rearward direction and to prevent relative movement in a direction transverse to said forward-rearward direction.

7. The oral dental appliance of claim 6, wherein said one of said clips includes indicia indicative of said forward-rearward position of said clips relative to each other.

8. The oral dental appliance of claim 4, wherein said clips each include interlocking notches therebetween configured to interlock at said adjustable position of said clips relative to each other.

9. The oral dental appliance of claim 4, wherein said fastener includes a bolt and a nut.

10. The oral dental appliance of claim 4, wherein:
one of said clips defines a slot elongated in the direction of said forward and rearward movement;
the other of said clips defines a bore aligned with said slot when the clips are connected by said fastener; and
said fastener includes an elongated fastener configured to extend through said slot and said bore when the clips are connected.

11. The oral dental appliance of claim 10 wherein said elongated fastener is a bolt and said fastener includes a nut embedded within said other of said clips, said nut defining said bore and configured for threaded engagement with said bolt.

12. The oral dental appliance of claim 10, wherein said slot has a length sized so that said adjustable forward-rearward position of said clips relative to each other spans 10 mm.

13. The oral dental appliance of claim 12, wherein said slot and said bore are arranged in said one and said other of said clips, respectively, so that said clips can be moved between a first position in which in which said rear end of said upper clip is rearward of said rear end of said lower clip and a second position in which said rear end of said upper clip is forward of said rear end of said lower clip.

14. The oral dental appliance of claim 13, wherein in said first position said rear end of said upper clip is 7 mm rearward of said rear end of said lower clip and in said second position said rear end of said upper clip is 3 mm forward of said rear end of said lower clip.

15. The oral dental appliance of claim 4, wherein the clips are engageable to the respective trays so that the respective trays can move relative to the clips in a direction transverse to said forward-rearward direction.

16. The oral dental appliance of claim 15, wherein:
the trays each include a back wall defining a slot elongated in the transverse direction; and
the clips each include a projection arranged to be engaged within said slot in the corresponding tray when said clips are engaged to the respective trays, said projection sized to slide along said slot in the transverse direction.

17. The oral dental appliance of claim 16, wherein said slot has a length of 2.0 mm.

18. The oral dental appliance of claim 16, wherein the clips each include a resiliently deflectable elongated arm connected at one end to the rear end of the clip and including said projection at the opposite end.

19. An adjustment mechanism for an adjustable clip assembly, the clip assembly including an upper clip, a lower clip and a fastener for connecting the upper clip and the lower clip and for fastening the upper clip and the lower clip together, the adjustment mechanism comprising:
a surface defining a recess for receiving one of the clips in a fixed position, with the other of the clips connected to said one of the clips;
a dial on said surface, said dial bearing indicia indicative of the forward-rearward position of the other of the clips relative to said one of the clips;
an indicator needle including a tip at one end aligned with said dial, a guide post at the opposite end, and a pivot post between said tip and said guide post, said pivot post pivotably mounted on said surface; and
a generally planar tool adapted to be in slidable contact with said surface, said tool defining a housing for receiving said other of the clips for movement with said tool, and defining an elongated slot for slidably receiving said guide post,
whereby movement of said tool in a forward-rearward direction;
moves said other of the clips relative to said one of the clips received in said recess; and
moves said guide post within said elongated slot such that the movement of said guide post causes rotation of said indicator needle about said pivot post so that said tip moves relative to said dial to an indicia indicative of the forward-rearward position of the other of the clips relative to said one of the clips.

20. The adjustment mechanism of claim 19, wherein said tool includes an opening in said housing for access to said fastener to fasten the upper clip and the lower clip together at the forward-rearward position.

21. The adjustment mechanism of claim 19, further comprising a case for receiving the clip assembly, said case including a lid with said surface.

22. The adjustment mechanism of claim 19, wherein:
said surface includes a pair of posts projecting therefrom, said posts aligned in said forward-rearward direction; and
said tool defines a pair of guide slots arranged to be seated on said pair of posts to limit the slidable contact with said surface to said forward-rearward direction.

23. The adjustment mechanism of claim 19, wherein the distance along said indicator needle between said tip and said pivot post is at least three times the distance along said indicator needle between said pivot post and said guide post.
